(19) United States
(12) Patent Application Publication
(10) Pub. No.: US 2008/0050445 A1
(43) Pub. Date: Feb. 28, 2008

(54) Niosome-Hydrogel Drug Delivery

(75) Inventors: Norma Alcantar, Tampa, FL (US); Kristina Dearborn, Tampa, FL (US); Michael VanAuker, Wesley Chapel, FL (US); Ryan Toomey, Tampa, FL (US); Elizabeth Hood, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/737,271

(22) Filed: Apr. 19, 2007

Related U.S. Application Data
(60) Provisional application No. 60/745,126.
(60) Provisional application No. 60/807,122, filed on Jul. 12, 2006.

(51) Int. Cl. A61K 9/50

(52) U.S. Cl. 424/490

(57) ABSTRACT

Taught herein is a drug-delivery system that includes encapsulating a therapeutic drug in a nanoparticle vesicle that is then embedded into a hydrogel network. The system allows for enhanced, two-fold control over the release rate of the drug. This technology will be particularly advantageous in treating malignant cancer cells such as those found in the brain. The invention will allow for decreased side effects and increased survival time in patients. This invention opens the door to other technological applications that require controlled release of chemical substances.

### PRIORITY CLAIM

This application claims priority from U.S. Provisional Application No. 60/807,122 filed on Jul. 12, 2006 entitled “Drug-Encapsulated Niosomes Embedded in Biodegradable Polymer Hydrogels for Use in Drug Delivery Applications” and U.S. Provisional Application No. 60/745,126 filed Apr. 19, 2006 entitled “Drug-Encapsulated Niosomes Embedded in Biodegradable Polymer Hydrogels for Use in Drug Delivery Applications.”

### FIELD OF INVENTION

This invention relates to drug delivery. More particularly, this invention relates to controlling the release rate of a therapeutic drug using nanoparticle vesicles embedded in hydrogel networks.

### SUMMARY OF INVENTION

This invention incorporates encapsulating a therapeutic drug in a nanoparticle vesicle that will be embedded into a hydrogel network and will allow for double enhanced control over the release rate of the drug to malignant cancer cells. The invention will allow for decreased side effects and increased survival time in patients. This invention opens the door to other technological applications that require controlled release of chemical substances.

This invention will be of great interest for use in drug delivery devices, particularly for the treatment of diseases such as cancer. This invention is designed for applications at physiological temperature and pH conditions. For example, the drug delivery device could be used by a surgeon after major brain surgery to be implanted in the tumor cavity of cancer patients. The invention could be used in applications where radiation or other harsh treatment methods are not a plausible option and injection-diffusion delivery is the best alternative.

The invention is also not limited to drug delivery for brain tumor patients. It could also be modified and applicable to any illness in which a site-specific delivery of active ingredient is needed. Other cancers such as breast cancer are prime candidates for the invention because the invention directly targets the infectious site. This invention is also useful in other commercial applications that require the controlled release of a product such as in pesticides or water treatment applications.

Applications for this technology include brain cancer patients who have been diagnosed with glioblastoma multiforme (glioma). These patients are in need of alternative chemotherapy treatment that is less harsh than traditional chemotherapy techniques used for other types of cancer. The sensitivity and delicate nature of the human brain makes this invention desirable to brain cancer patients and an excellent potential alternative to treatment.

Current brain tumor drug delivery methods create severe adverse side effects and are not efficient for long-term survival. A patient diagnosed with a glioma has less than one year even with standard therapy. This technology provides an increased survival time and decreased side effects in these patients due to the direct control and targeting that the design proposes. Other areas for application include any patient in need of site-specific intervention.

An embodiment of the invention includes a drug delivery medium comprising a drug-encapsulated niosome embedded in a biodegradable polymer hydrogel. The hydrogel properties are preselected in accordance with a desired release rate for the niosomes and may include cross-link density, pH-sensitivity and temperature sensitivity. In addition to hydrogel properties, niosome constituents are preselected in accordance with a desired release rate for the encapsulated drug and may include polysorbates, cholesterols, and dicetyl phosphates (i.e., polyoxyethylene (61) sorbitan monostearate). The concentration of the preselected niosome constituents are varied in accordance with the desired release rate for the encapsulated drug.

A plurality of biodegradable polymer hydrogel layers may sandwiched together thereby forming a gradient network to package vesicles in varying microenvironments to tune release rates from an identical population of vesicles wherein adjoining hydrogel layers have distinct properties. The distinct properties of the hydrogel layers may include cross-link density, pH-sensitivity and temperature sensitivity.

### BRIEF DESCRIPTION OF DRAWINGS

For a fuller understanding of the invention, reference should be made to the following detailed description, taken in connection with the accompanying drawings, in which:

FIG. 1 is a conceptual illustration of passive packaging for vesicles of different sizes, cargo and/or membrane composition.

FIG. 2 is a conceptual illustration of a gradient network packaging vesicles in varying microenvironments.

FIG. 3 is a conceptual illustration of stimuli-responsive networks to control microenvironment and release properties of vesicles.

FIG. 4 is a data plot demonstrating linear release of molecules retained in niosomes according to an embodiment of the invention.

### DETAILED DESCRIPTION OF THE PREFERRED EMBODIMENT

An embodiment of this invention addresses the problem of on-site brain tumor treatment by providing a controlled release of drugs to malignant cancer cells. The invention improves on how medication is administered to patients and reduces adverse side effects associated with over-dosage. Benefits to the patient include offering more effective techniques of eliminating cancer cells that may still be present after surgery and thus providing better health conditions following treatment. Alternative embodiments of this invention are useful in the controlled release of chemical substances for engineering applications such as battery packaging and antifouling agents.

The emerging field of nanoparticle material science has become increasingly important in the biomedical and bioengineering fields owing to the ability to incorporate nanostructured materials in the design of life-saving technologies. The treatment of malignant cancer cells after major brain surgery is one such area that could benefit from the application of nanostructured materials. Traditional cancer treatments, such as chemotherapy, are not practical options in this situation due to the sensitivity and care that must be taken when dealing with matters of the brain.

The underlying research that led to the conception and reduction to practice of the present invention was aimed at designing a technique of drug delivery to brain tumor cells which was efficient and effective by controlling the release rate of the drug. The smart-packaging technique that this research produced incorporates a double control mechanism that allows for the maximum determination of the release rate.

Fluorescent carboxyfluoroscein (CF) dye is encapsulated in a non-ionic surfactant vesicle, or niosome, and embedded in a biodegradable chitosan polymer hydrogel. Carboxyfluoroscein dye is used as a tracer dye and indicates the release of the drug from the system. Chitosan is a temperature and −pH sensitive polymer that will begin to gel and form the hydrogel network at physiological conditions (T=37° C.; pH=6.2). This feature allows for the direct formation of the smart-packaging system at the point of contact within the brain cavity, which eliminates the risk of contamination or interference from other secondary sources. A unique property of the chitosan polymer is its ability to be molded into any shape desired. This allows for the cavity-specific shape of the system to be made, thus eliminating the risk of unevenly distributing the drug. The release rate of the CF dye was determined for the system at various volumes for various time intervals. The concentration of the CF dye was determined using fluorescence spectrometry. CF dye has an excitation/emission range of 492 nm/514 nm.

It was determined that the release rate was able to be controlled using the niosome/hydrogel system and that the smart-packing method is a viable technique useful for treatment of cancer cells in brain tumor cavities. The CF dye release rate from the niosome was quantified as well as the release rate from the chitosan hydrogel as the polymer decomposed. Because CF dyes have similar molecular weights to chemotherapy drugs it validates the advanced control for the release rate of drugs using nanoparticle materials. This release system in addition to the smart-packaging system for the brain decreases the toxicity of medication to other parts of the body, increase direct utilization of the drug, increase the survival time of the patients, and improves their quality of life.

This invention consists of two main components: the niosome and the hydrogel. The niosome is a non-ionic surfactant vesicle that is similar to that of a liposome. It is composed of synthetic amphiphilic surfactants and cholesterol that make up a bilayer membrane and is able to entrap hydrophilic solutions in the aqueous core and hydrophobic solutions in the non-polar membrane. The advantage of using the niosomes as opposed to the liposomes is that the synthetic niosomes have shown to be more chemically stable as vesicles, they are easier to transport and store, they are less expensive, and they have been shown to increase the blood brain barrier permeability. The niosomes are prepared using cholesterol, dicetyl phosphate (DCP), and a surfactant such as sorbitan monosterate. The niosomes are synthesized through thin film hydration and sonication.

A fluorescent dye is encapsulated in the core of the niosomes and is used as a tracer dye that allows for the detection of dye during in vitro experiments. The dye that is used in this invention is 5(6)-carboxyfluorescein. The second component of the invention is the hydrogel. A hydrogel is a water-soluble polymer membrane that consists of crosslinked macromolecules. The crosslinked characteristic makes hydrogels resistant to dissolution and ideal for encapsulating smaller particles such as niosomes.

The hydrogel component provides three unique features for the system: 1) It prevents free niosomes from circulating throughout the body that may cause underutilization to the active sites; 2) It provides a safe place where the niosomes will be preserved until needed because of their ability to be altered according to a desired functionality; 3) It provides another control opportunity for the drug due to the release rate of the drug through both the niosome and the hydrogel. The hydrogel is prepared by using a chitosan/glycerophosphate (GP) thermosensitive polymer solution that begins to form a gel at physiological conditions of 37° C. and a pH of 6.2. Chitosan is a biodegradable and biocompatible polymer.

Glycerophosphate neutralizes the chitosan solution so that the gellation process will occur only when the temperature is raised to 37° C. and the solution will remain in the liquid state until this condition is met. The niosomes could be incorporated into the hydrogel network by the use of simple physical techniques such as mixing on-site in the brain tumor cavity.

This invention includes the novel concept of a double control mechanism which will allow for enhanced control over the release rate of the drug. The “package-within-a-package” is an idea that has not yet been explored by those in the drug delivery community and has the potential for revolutionizing how therapeutic drugs can be administered.

In a general embodiment the invention, hydrogel structures that embed niosomes, has particular utility for three types of uses. The first use is passive packaging for vesicles of different sizes, cargo, or membrane composition. This would allow for the embedment of a drug that may come in the form of varying size and shape. This could also allow for multiple drugs to be embedded in the sample hydrogel network. The second use takes advantage of the gradient network to package the vesicles in varying microenvironments. This characteristic allows one to manipulate the release rate of the drug by altering chemical and physical properties such as cross-link density. Finally, the stimuli-responsiveness of the networks allows one to control the microenvironment and the release rate of the niosomes. These examples illustrate how the hydrogel/niosome network will behave at certain temperatures and pHs and how drug delivery will be conducted and controlled.

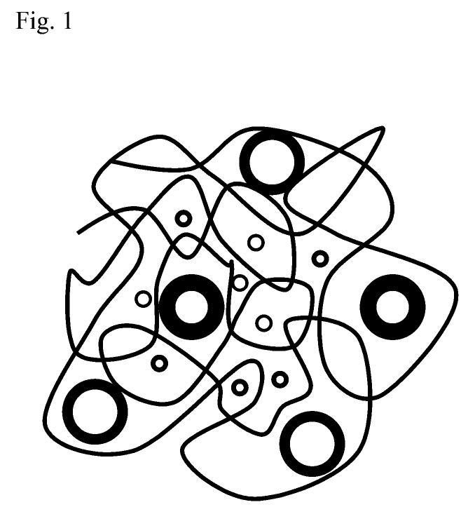

FIG. 1 demonstrates passive packaging for vesicles of different sizes, cargo, or membrane composition. This allows for the embedment of a drug with varying size and shape. This also permits multiples drugs to be embedded in the same hydrogel network.

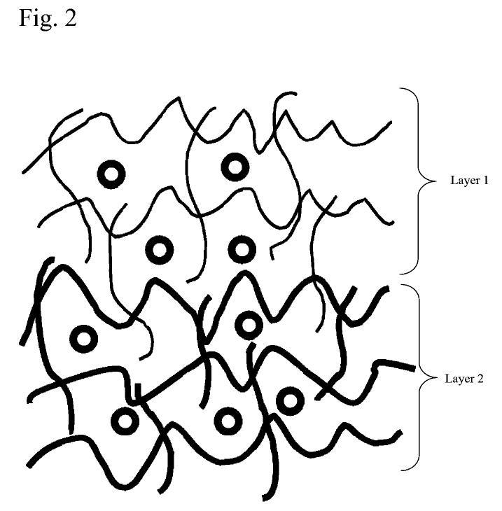

FIG. 2 demonstrates the gradient network to package the vesicles in varying microenvironments. This characteristic allows one to manipulate the release rate of the drug by altering chemical and physical properties such as cross-link density.

Viscoelasticity may be modulated to tailor surfaces that support large loads with little deformation.

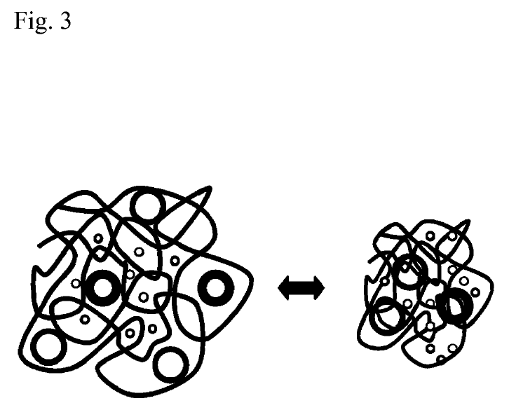

FIG. 3 demonstrates the stimuli-responsiveness of the networks that allow one to control the microenvironment and the release rate of the niosomes. This illustrates how the hydrogel/niosome network behaves at certain temperatures and pH levels.

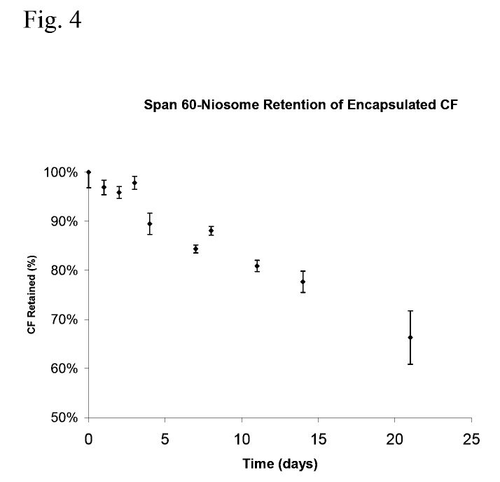

The data shown in FIG. 4 represents the amount of carboxyfluorescein dye that was retained in Span 60 niosomes after gel exclusion chromatography separation versus time. This demonstrates that the dye is being released in a linear manner. The dye is not just being released randomly. This indicates that one would be able to control for the release rate of they dye from the niosomes by manipulating other properties of the system.

Table 1 is a comparison of CF encapsulation over time with varying mol % of Tween 61 included in Span 60 niosomes. CF concentration was monitored for 14 days for all samples except 0 and 100% which were monitored for 9 days. It represents the amount of carboxyfluorescein dye retained in the niosomes by changing the mole percentage of Tween 61 versus time.

TABLE 1

| % Tween | % CF Retained over time |
|---|---|
| 0 | 88.0% |
| 1 | 85.9% |
| 3.25 | 84.8% |
| 5.5 | 89.1% |
| 10 | 88.0% |
| 100 | 62.2% |

Tween 61 is a component of the niosomes, and tests of the invention indicates that varying the concentration of one of the niosome components can change the release of the dye. By increasing the concentration of the Tween 61, the percentage of dye that is released is increased. This information can be combined with similar information for the concentration of the other components of the niosomes, i.e. cholesterol, dicetyl phosphate, to optimize the release of the drug.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A drug delivery medium comprising a drug-encapsulated niosome embedded in a biodegradable polymer hydrogel.

2. The medium of claim 1 wherein the hydrogel properties are preselected in accordance with a desired release rate for the niosomes.

3. The medium of claim 2 wherein the hydrogel properties are selected from the group consisting of cross-link density, pH-sensitivity and temperature sensitivity.

4. The medium of claim 1 wherein niosome constituents are preselected in accordance with a desired release rate for the encapsulated drug.

5. The medium of claim 4 wherein the niosome constituents are selected from the group consisting of polysorbates, cholesterols, and dicetyl phosphates.

6. The medium of claim 4 wherein the niosome constituents include polyoxyethylene (61) sorbitan monostearate.

7. The medium of claim 4 wherein the concentration of the preselected niosome constituents are varied in accordance with the desired release rate for the encapsulated drug.

8. The medium of claim 7 wherein the concentration of polyoxyethylene (61) sorbitan monostearate is varied in accordance with the desired release rate for the encapsulated drug.

9. A drug delivery medium comprising drug-encapsulated niosomes embedded in a plurality of biodegradable polymer hydrogel layers, wherein adjoining layers have distinct properties.

10. The drug delivery medium of claim 9 wherein the distinct properties of the hydrogel layers are selected from the group consisting of cross-link density, pH-sensitivity and temperature sensitivity.

11. A drug delivery medium for application within a brain cavity comprising a drug-encapsulated niosome embedded in a biodegradable polymer hydrogel, the hydrogel comprising a biodegradable chitosan polymer.
